(19) United States
(12) Patent Application Publication
(10) Pub. No.: US 2007/0248629 A1
(43) Pub. Date: Oct. 25, 2007

(54) PHARMACEUTICAL FORMULATIONS FOR IONTOPHORETIC DRUG DELIVERY

(76) Inventors: PHILLIP M. FRIDEN, BEDFORD, MA (US); BIRESWAR CHAKRABORTY, ANDOVER, MA (US); DINA BERKOVITZ, Newton, MA (US)

Correspondence Address: ELMORE PATENT LAW GROUP, PC, 209 MAIN STREET, N. CHELMSFORD, MA, 01863, US

(21) Appl. No.: 11/737,466

(22) Filed: Apr. 19, 2007

Related U.S. Application Data
(60) Provisional application No. 60/793,673, filed on Apr. 20, 2006.

(51) Int. Cl. A61K 31/522; A61K 9/14

(52) U.S. Cl. 424/400; 424/488; 514/263.31; 424/487

(57) ABSTRACT

The present invention provides pharmaceutical formulations suitable for iontophoresis that provide enhanced iontophoretic delivery of acyclovir (ACV) to at least one target tissue. The present invention also provides methods of treating viral infection in at least one target tissue of a patient by iontophoretically delivering a formulation of the invention to the infected target tissues of the patient.

### RELATED APPLICATION

This application claims the benefit of U.S. Provisional Application No. 60/793,673, filed on Apr. 20, 2006. The entire teachings of the above application are incorporated herein by reference.

### BACKGROUND OF THE INVENTION

An iontophoretic delivery system is, for example, a drug delivery system that releases drug at a controlled rate to the target tissue upon application. The advantages of systems wherein drug is delivered locally via iontophoresis are the ease of use, being relatively safe, and affording the interruption of the medication by simply peeling off or removing from the skin whenever an overdosing is suspected. The total skin surface area of adult is about 2 m². In recent years iontophoretic delivery of drugs has attracted wide attention as a better way of administering drugs for local as well as systemic effects. The design of iontophoretic delivery systems can usually be such that the side effects generally seen with the administration of conventional dosage forms are minimized.

Iontophoresis has been employed for many years as a means for applying medication locally through a patient's skin and for delivering medicaments to the eyes and ears. The application of an electric field to the skin is known to greatly enhance the ability of the drugs to penetrate the target tissue. The use of iontophoretic transdermal delivery techniques has obviated the need for hypodermic injection for some medicaments, thereby eliminating the concomitant problems of trauma, pain and risk of infection to the patient.

Iontophoresis involves the application of an electromotive force to drive or repel ions through the dermal layers into a target tissue. Particularly suitable target tissues include those adjacent to the delivery site for localized treatment. Uncharged molecules can also be delivered using iontophoresis via a process called electroosmosis.

Regardless of the charge of the medicament to be administered, an iontophoretic delivery device employs two electrodes (an anode and a cathode) in conjunction with the patient's skin to form a closed circuit between one of the electrodes (referred to herein alternatively as a “working” or “application” or “applicator” electrode) which is positioned at the site of drug delivery and a passive or “grounding” electrode affixed to a second site on the skin to enhance the rate of penetration of the medicament into the skin adjacent to the applicator electrode.

Researchers have investigated the potential for iontophoresis facilitated transdermal delivery of acyclovir (ACV). Lashmar and Manger, International Journal of Pharmaceutics 111(1994) 73-82, describe the use of the penetration enhancers sodium lauryl sulfate, an anionic surfactant and centrimide, a cationic surfactant, in conjunction with cathodal and anodal iontophoretic delivery of ACV to enhance iontophoretic permeation. Volpato et al. Pharmaceutical Research, 12 (1995) 1623-1627, describe studies aimed at determining the mechanisms responsible for transdermal delivery of ACV in vitro. Stagni et al. International Journal of Pharmaceutics 274 (2004) 201-211 compare the pharmokinetics of ACV in skin and plasma after delivery of ACV by iontophoresis, IV bolus and topical ointment administration in rabbits. Iontophoretic delivery of a standard ACV sodium for injection formulation showed a marked increase in the delivery rate of ACV to the rabbit skin over a commercial ACV topical formulation. It would be desirable to have stable formulations of ACV that possess good to excellent delivery characteristics of ACV to a target tissue by iontophoresis.

### SUMMARY OF THE INVENTION

The present invention provides pharmaceutical formulations suitable for iontophoresis that provide enhanced iontophoretic delivery of ACV to at least one target tissue. The formulations are further characterized by good to excellent stability. The present invention also provides methods of treating viral infection in at least one target tissue of a patient by iontophoretically delivering a formulation of the invention to the infected target tissue of the patient.

### BRIEF DESCRIPTION OF THE FIGURES

FIG. 1 illustrates the relative penetration of formulations according to the invention and controls via microdialysis study.

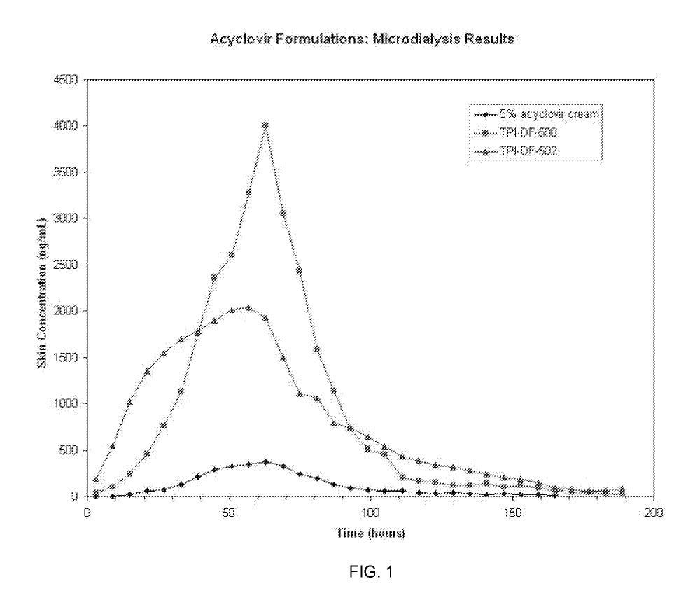

FIG. 2 illustrates the penetration of acyclovir with glycerin versus propylene glycol via rabbit microdialysis study.

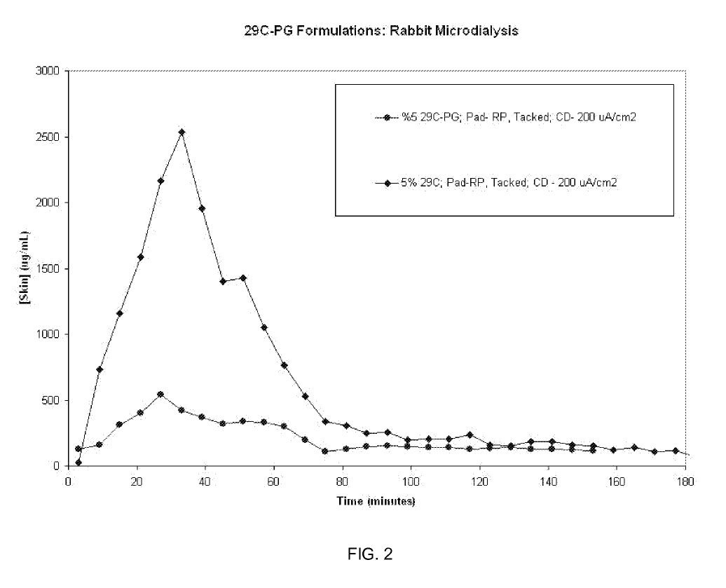

FIG. 3 illustrates the solubility of acyclovir with various levels of glycerin at each neutralization level.

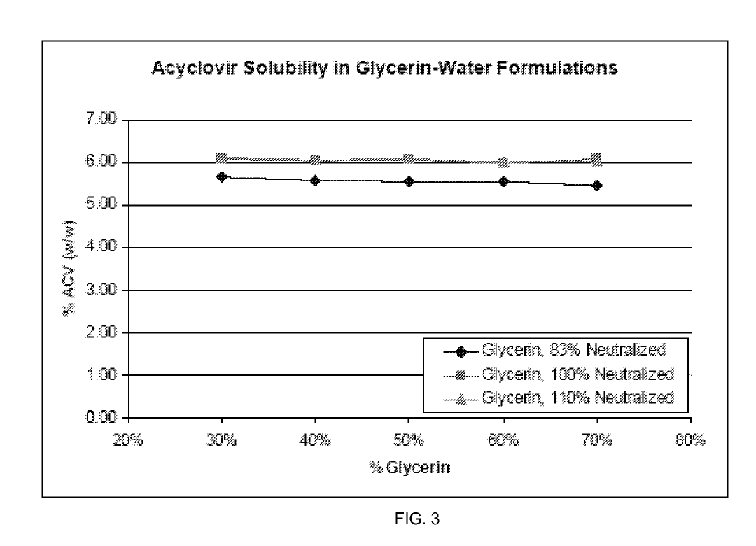

FIG. 4 illustrates the solubility of acyclovir with various levels of propylene glycol at each neutralization level.

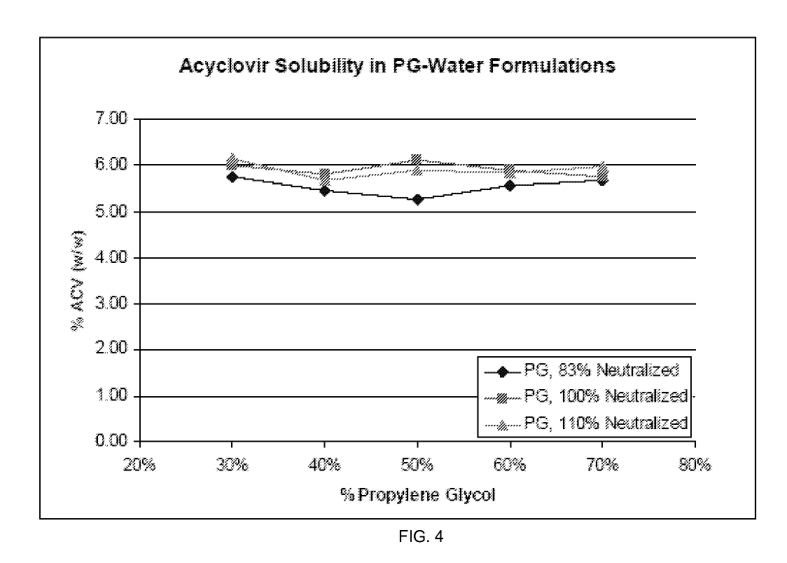

FIG. 5 shows the plot of the measured pH for each glycerin gel versus glycerin content.

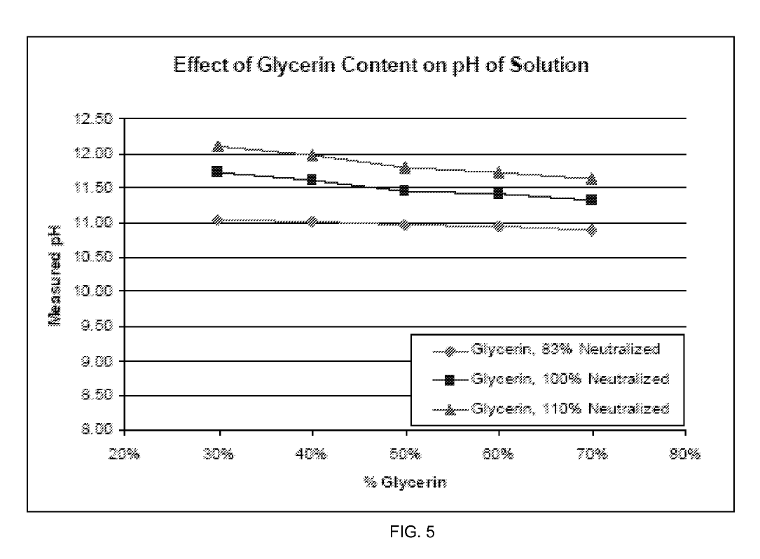

FIG. 6 shows the plot of the measured pH for each propylene glycol gel versus propylene glycol content.

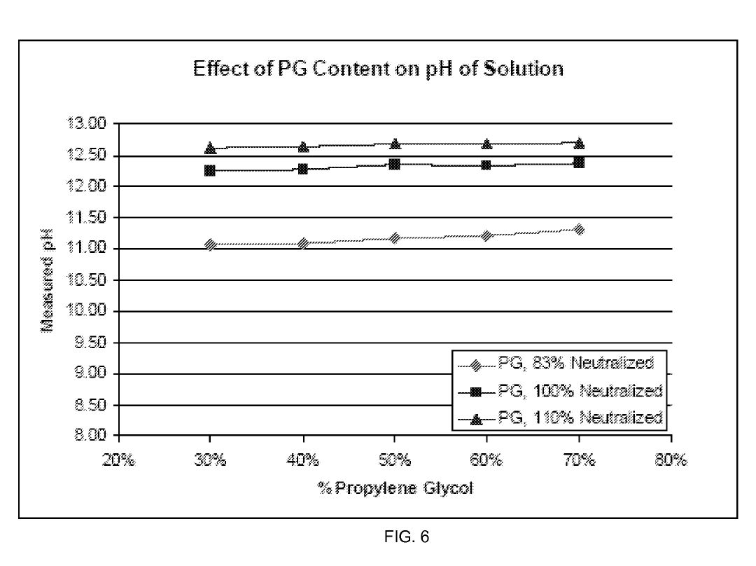

FIG. 7 shows the in vivo results of the active and passive delivery for both the cream and the 5% pH 11 glycerin gel.

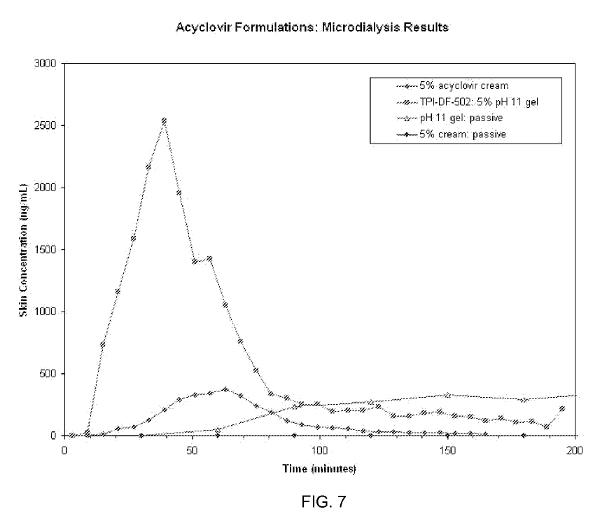

### DETAILED DESCRIPTION OF THE INVENTION

In one aspect, the invention provides pharmaceutical formulations that are suitable for iontophoresis and that deliver therapeutic levels of ACV to a patient for treating a viral infection in at least one target tissue of a patient, preferably a human patient, in need of treatment. A formulation of the invention is preferably a viscous formulation and comprises ACV, preferably the sodium salt thereof, and a pharmaceutically acceptable carrier or excipient, wherein the pH of the formulation is at least about 10. Alternatively or additionally, the formulation of the invention is a viscous formulation comprising a soluble ACV salt and a pharmaceutically acceptable carrier or excipient, substantially free of insoluble ACV. As used herein, the term “viscous formulation” includes colloidal and gel formulations. Alternatively or additionally, the formulation of the invention comprises ACV and a pharmaceutically acceptable carrier or excipient, wherein the pH of the formulation is at least about 10 further characterized by good to excellent stability properties. As used herein, a “stable formulation” includes formulations wherein the acyclovir remains in a soluble form, without substantial degradation, for at least 5 days while stored at temperatures between 5 and 30° C. As used herein, the term “pharmaceutically acceptable carrier or excipient” means any non-toxic, diluent or other formulation auxiliary that is suitable for use in iontophoresis. Examples of pharmaceutically acceptable carriers or excipients include but are not limited to: diluents such as water, or other solvents, cosolvents; solubilizing agents such as sorbital and glycerin; buffers such as, for example, phosphate buffer solutions; pharmaceutically acceptable bases; and viscosity modulating agents such as cellulose and its derivatives.

In one embodiment, the formulation is not ACV sodium for injection. In another embodiment, the formulation is a gel formulation.

As used herein the term “target tissue” includes the patient's dermis, epidermis, nails, mucocutaneous membranes including, but not limited to, the eye and the body cavity and canal sites such as mouth, ear, nose, vagina, and rectum.

In one preferred embodiment, the invention provides a pharmaceutical formulation suitable for iontophoresis comprising ACV the pH of the formulation is at least 10. The ACV can be added in its salt form or as a free base. In the latter embodiment, an ACV salt can be formed in situ. Throughout this specification, one of ordinary skill in the art can readily discern or determine whether the ACV referred to is in its free base or salt form. In general, it is desirable to add or produce a soluble ACV salt in the formulations of the invention. The formulation may be a viscous and/or stable formulation or a solution. In one embodiment, the formulation comprises a buffer, such as a phosphate buffer. In one embodiment, the formulation comprises about 0.3 to about 10 weight percent, preferably between about 3 to about 6 weight percent, of ACV and/or about 1 to about 10 weight percent of buffer. Alternatively, the formulation is buffer free, which has the advantage of fewer competing ions during iontophoresis.

The invention is based, in part, on the discovery that the selection of glycerin as a solubilizing agent resulted in substantially improved uptake, as compared to propylene glycol. Without being bound by theory, it is believed that his effect is due to glycerin's improved hydrating properties. In one preferred embodiment, the invention provides a pharmaceutical formulation suitable for iontophoresis comprising ACV, hydrating agent, such as glycerin, and a solvent (e.g., water), wherein the pH of the formulation is at least 10. The formulation may be a viscous formulation. In one embodiment, the formulation comprises about 3 to about 6 weight percent of ACV (preferably about 4%) and about 10 to about 80 weight percent of glycerin (preferably about 50%). The formulation may comprise about 20 to about 99 weight percent of water (preferably about 40%).

In one preferred embodiment, the invention provides a pharmaceutical formulation suitable for iontophoresis comprising ACV, glycerin and one or more buffers, wherein the pH of the formulation is at least 10 and the formulation is in the form of a viscous formulation. Preferably the buffer is a phosphate buffer solution and is added in an amount of about 1 to about 10 weight percent of Na₂HPO₄/Na₃PO₄ (preferably about 2%). In one embodiment, additional base is added to the formulation. For example, NaOH, e.g., 5N NaOH can be added in an amount sufficient to achieve the desired pH. For example in one embodiment, about 4 weight percent of 5.0 N NaOH is added.

In one aspect, the invention provides a pharmaceutical formulation suitable for iontophoresis having a pH of at least 10 comprising ACV and a pharmaceutically acceptable carrier or diluent, wherein the formulation is a viscous formulation having a viscosity of greater than about 400 cp, such as at least about 500 cp at 25° C. In one embodiment, the viscosity is about 590 cp. A viscosity modifying agent can be added to the formulation to achieve the desired viscosity. The pharmaceutically acceptable carrier or excipient may comprise about 0.1 to 10 weight percent of a viscosity modulating agent.

In one aspect, the invention provides a pharmaceutical formulation suitable for ionotophoresis that may further comprise at least one antioxidant, stabilizer, chelator, preservative, aldehyde scavenger or mixture thereof Preferably, the excipients should be uncharged so as not to compete with the acyclovir transport.

The term “antioxidant” is intended to mean an agent which inhibits oxidation and thus is used to prevent the deterioration of preparations by the oxidative process. Such compounds include by way of example and without limitation, acetone, sodium bisulfate, ascorbic acid, alpha-tocopherol, ascorbyl palmitate, citric acid, butylated hydroxyanisole, butylated hydroxytoluene, hydrophosphorous acid, monothioglycerol, propyl gallate, sodium ascorbate, sodium citrate, sodium sulfide, sodium sulfite, sodium bisulfite, sodium formaldehyde sulfoxylate, thioglycolic acid, sodium metabisulfite, EDTA (edetate), pentetate and others known to those of ordinary skill in the art.

The term “stabilizer” is intended to mean a compound used to stabilize a therapeutic agent against physical, chemical, or biochemical process that would otherwise reduce the therapeutic activity of the agent. Suitable stabilizers include, by way of example and without limitation, albumin, sialic acid, creatinine, glycine and other amino acids, niacinamide, sodium acetyltryptophonate, zinc oxide, sucrose, glucose, lactose, sorbitol, mannitol, glycerol, polyethylene glycols, sodium caprylate and sodium saccharin and others known to those of ordinary skill in the art.

The term “chelator” as used herein refers to a molecule that binds metal ions, usually by binding to two or more complexing groups within the molecule. Chelators are well known in the art, and include certain proteins and polypeptides, as well as small molecules such as ethylenediaminetetraacetic acid (EDTA), ethylene glycol-bis(β-aminoethyl ether)-N,N,N′,N′-tetraacetic acid (EGTA), nitrilotriacetic acid, oxalate, citric acid, 1,2-diaminocyclohexane-N,N,N′N′-tetracetic acid, 4,5-dihydroxybenzene-1,3-disulfonic acid, pyrocatechol-3,5-disulfonate, salicylic acid, 5-sulfosalicylic acid, xylenol orange, aurintricarboxylic acid, 2,2′-pyridyl ethylene diamine, glycine, 8-hydroxyquinoline-5-sulfonic acid, lactic acid, 1,10-phenanthroline, pyridine, pyridine-2,6-dicarboxylic acid, 8-quinolinol, succinic acid, tartaric acid, thioglycolic acid, 1,1,1-trifluoro-3,2′-thenolyacetone, triethylene tetramine and the like.

The preservatives include antimicrobial agents that kill and/or inhibit the proliferation and/or growth of microbes, particularly bacteria, fungi and yeast. Preservatives can be synthetic compounds, semisynthetic compounds, and naturally produced compounds. Suitable dermatologically absorbable preservatives include erythromycin, bacitracin, zinc bacitracin, polymycin, neomycin, chloramphenicol, tetracycline, sulfacetamide, minocycline, clindamycin, doxycycline, undecylenic acid and salts thereof, propionic acid and salts thereof, caprylic acid and salts thereof, ciprofloxacin, cephlasporins, benzoic acid, ciclopiroxolamine, clotrimazole, econazole nitrate, metronizadole, miconazole nitrate, ketacanazole, oxiconazole, tolnaftate, benzalkonium chloride, parabens, methyl paraben, benzethonium chloride, Neolone 950, sodium benzoate, sodium bisulfite, phenol, alkyl esters of parahydroxybenzoic acid, o-phenylphenol benzoic acid and salts thereof, boric acid and salts thereof, sorbic acid and salts thereof, chlorobutanol, benzyl alcohol, thimerosal, phenylmercuric acetate and nitrate, nitromersol, and cetylpyridinium chloride.

The term “aldehyde scavenger” as used herein is a substance that reacts with an aldehyde to form a neutralized aldehyde that has decreased ability to form adducts with the amino groups of acyclovir and that does not itself react with acyclovir. Aldehyde scavengers include, for example, substances that contain primary amine groups that react with aldehyde functional group(s). Aldehyde scavengers also include sulfites. Suitable aldehyde scavengers include,but are not limited to, urea, methionine and methionamide.

The term “base” is used in its traditional sense, i.e., a substance that disassociates in water to produce hydroxide ions. Any base may be used provided that the compound provides free hydroxide ions in the presence of water. Such bases include inorganic or organic pharmaceutically acceptable bases. Preferred inorganic bases include inorganic hydroxides, such as alkali metal hydroxides, carbonates, inorganic oxides, inorganic salts of weak acids and combinations thereof. Preferred organic bases are nitrogenous bases, such as amines and quaternary ammonium bases. In one preferred embodiment, the base is NaOH.

The terms “neutralized” or “neutralization” refer to the formation of an acyclovir salt. In a preferred embodiment, the salt is sodium acyclovir.

Unless otherwise stated, the weight percentage of acyclovir refers to the free base form of the compound, as compared to the salt form. The amount of “soluble” acyclovir or the weight percent of the ACV salt in the formulation can be readily determined by the person of ordinary skill in the art.

The viscosity of the viscous formulation may be controlled by a viscosity modulating agent. A viscosity modulating agent includes any agent that is capable of modulating the viscosity of a gel. Viscosity modulating agents useful in the practice of the invention include but are not limited to, ionic and non-ionic, high viscosity, water soluble polymers; crosslinked acrylic acid polymers such as the “carbomer” family of polymers, e.g., carboxypolyalkylenes that may be obtained commercially under the Carbopol® trademark; hydrophilic polymers such as polyethylene oxides, polyoxyethylene-polyoxypropylene copolymers, and polyvinylalcohol; cellulosic polymers and cellulosic polymer derivatives such as hydroxypropyl cellulose, hydroxyethyl cellulose, hydroxypropyl methylcellulose, hydroxypropyl methylcellulose phthalate, methyl cellulose, carboxymethyl cellulose, and etherified cellulose; gums such as tragacanth and xanthan gum; sodium alginate; gelatin, hyaluronic acid and salts thereof, chitosans, gellans or any combination thereof. If a uniform gel is desired, dispersing agents such as alcohol, sorbitol or glycerin can be added, or the gelling agent can be dispersed by trituration, mechanical mixing, or stirring, or combinations thereof. In one embodiment, the viscosity enhancing agent can also provide the base, discussed above.

In one preferred embodiment, the viscosity modulating agent is cellulose that has been modified such as by etherification or esterification. One such etherified cellulose polymer is sold under the trademark Natrosol® (Hercules-Aqualon, Wilmington, Del.).

Additionally, a surfactant or wetting agent can be added to facilitate application or wetting of the formulation to the iontophoresis pad material, or drug cartridge pad. Examples of suitable surfactants or wetting agents include surfactants such as polyoxyethylene hydrogenated castor oil 60, polyoxyethylenesorbitan monooleate, polyoxyethylenesorbitan monolaurate, polyoxyethylenelauryl ether, polyoxyethyleneoctyl phenyl ether, polyoxyethylenenonyl phenyl ether, polyoxyethylene polyoxypropylene glycol, polysorbate and saccharose aliphatic acid ester; saccharides such as glucose, maltose, fructose, galactose, mannitol, sorbitol, mannose, glucosamine, lactose, sucrose and trehalose; water-soluble cyclodextrins including natural cyclodextrins such as .alpha.-cyclodextrin, .beta.-cyclodextrin and .gamma.-cyclodextrin, water-soluble cyclodextrin derivatives having a substituent including hydroxypropyl, glycolyl, maltosyl, sulfate, phosphate, carboxyl, carboxymethyl, carboxymethylethyl and/or amino, and cyclodextrin polymers; water-soluble polymers such as starches, dextran, dextran sulfate, inulin and polyvinylpyrrolidone; and wetting agents such as glycerol, ethyleneglycol, polyethyleneglycol, propyleneglycol, butyleneglycol, urea, ethylurea, urea derivatives, methylpyrrolidone and pyrrolidone derivatives, may be exemplified.

In a preferred embodiment, an iontophoretic formulation of the present invention comprises about 4 weight percent of ACV; about 87 weight percent of sorbitol (70%); about 8 weight percent of 5.0 N sodium hydroxide; and about 1 weight percent of one or more viscosity modulating agents preferably one or more cellulosic polymers, optionally further comprising water.

In another preferred embodiment, an iontophoretic formulation of the present invention comprises about 4 weight percent of ACV; about 87 weight percent of sorbitol (70%); about 6 weight percent of 5.0 N sodium hydroxide; about 1 weight percent of one or more viscosity modulating agents preferably one or more cellulosic polymers; about 1 weight percent of Na₂HPO₄; and about 1 weight percent of Na₃PO₄, optionally further comprising water.

In another preferred embodiment, an iontophoretic formulation of the present invention comprises about 5 weight percent of ACV; about 48 weight percent of glycerin; about 5 weight percent of 5.0 N sodium hydroxide; about 1 weight percent of one or more viscosity modulating agents preferably one or more cellulosic polymers; and about 41 weight percent of water.

In another preferred embodiment, an iontophoretic formulation of the present invention comprises about 2 weight percent of ACV; about 93 weight percent of glycerin; about 2 weight percent of 5.0 N sodium hydroxide; about 1 weight percent of one or more viscosity modulating agents preferably one or more cellulosic polymers; and 2 weight percent of water.

In yet another embodiment, an iontophoretic formulation of the present invention comprises about 2 weight percent of ACV; about 49 weight percent of sorbitol (70%); about 2 weight percent of 5.0 N sodium hydroxide; about 1 weight percent of one or more viscosity modulating agents preferably one or more cellulosic polymers; and about 47 weight percent of water.

In one embodiment, the composition of Formulations B, C, D, E, and F are shown in Table 1:

TABLE 1

| Formulation | Acyelovir | H2O | Glycerin | Sorbitol (70%) | 5.0 N NaOH | pH | Natrosol 250 H | Na2HPO4 | Na3PO4 |
|---|---|---|---|---|---|---|---|---|---|
| 1849-B | | | | | | | | | |
| grams | 9.40 | 0 | 0 | 185.2 | 15.34 | 11.0 | 2.0 | 0 | 0 |
| % w/w | 4.44 | 0 | 0 | 87.4 | 7.24 | 11.0 | 0.94 | 0 | 0 |
| 1849-C | | | | | | | | | |
| grams | 9.35 | 0 | 0 | 184.0 | 12.70 | 11.0 | 2.0 | 1.70 | 2.88 |
| % w/w | 4.40 | 0 | 0 | 88.5 | 6.01 | 11.0 | 0.94 | 0.80 | 1.35 |
| 1849-D | | | | | | | | | |
| grams | 0.42 | 82.5 | 100.0 | 0 | 9.28 | 11.0 | 2.0 | 0 | 0 |
| % w/w | 4.63 | 40.7 | 49.0 | 0 | 4.58 | 11.0 | 0.98 | 0 | 0 |
| 1849-E | | | | | | | | | |
| grams | 4.20 | 3.70 | 0 | 192.8 | 4.61 | 10.8 | 2.0 | 0 | 0 |
| % w/w | 2.03 | 1.79 | 0 | 99.0 | 2.22 | 10.8 | 0.95 | 0 | 0 |
| 1849-F | | | | | | | | | |
| grams | 9.00 | 94.9 | 100.0 | 0 | 2.83 | 10.0 | 2.0 | 0 | 0 |
| % w/w | 1.48 | 46.9 | 49.4 | 0 | 1.4 | 10.5 | 1.0 | 0 | 0 |

The total cumulative amount of ACV penetrated per unit area of the skin during 4 to 8 hrs from 5 formulations were compared with that of the control (5% ACV). As shown in Table 2, Formulations B, C, D, and F all resulted in ACV penetration greater than the control formulation at 4 and 8 hours.

TABLE 2

| | | ACV | | |
|---|---|---|---|---|
| | | Concentration¹ | 4 hr² | 8 hr² |
| Formulation | Composition | mg/mL | (μg/cm²) | (μg/cm²) |
| 5% Cream | | | 2.2 | 4.4 |
| B | Sorbitol, | 56.1 | 5.5 | 10.3 |
| | pH 11 | | | |
| C | Sorbitol, | 64.1 | 10.6 | 26.3 |
| | pH 11, buffer | | | |
| D | Glycerin, | 42.7 | 4.0 | 12.2 |
| | pH 11 | | | |
| E | Sorbitol, | 35.2 | 3.2 | 4.4 |
| | pH 10.5 | | | |
| F | Glycerin | 15.2 | 6.3 | 13.6 |
| | pH 10.5 | | | |

¹By HPLC
²60 minute iontophoresis at a current density of 200 microamperes/cm².

In another preferred embodiment, representative formulations of the invention are listed in the Table 3 below:

TABLE 3

| Ingredients | TPI-DF-500 | TPI-DF-501 | TPI-DF-502 |
|---|---|---|---|
| Active Ingredient | | | |
| Acyclovir | 4.00 | 4.00 | 5.00 |
| Other | | | |
| Ingredients | | | |
| Glycerin | 50.00 | | 50.00 |
| Propylene glycol | | 50.00 | |
| Water, purified | 41.67 | 43.82 | 43.75 |
| Sodium hydroxide | 1.78 | 1.78 | 0.90 |
| Hydroxyethyl | 0.40 | 0.40 | 0.35 |
| cellulose | | | |

In a preferred embodiment, desirable solutions for iontophoresis have all the drug in solution and the concentration of the drug should not be too near the drug solubility limit. If the drug concentration is near the solubility limit small changes in temperature or composition can result in drug precipitation.

In yet another embodiment, the iontophoretic formulation of the present invention comprises from about 2 to about 6 weight percent ACV, glycerin and EDTA wherein the formulation has a pH above 10. In another embodiment, the formulation comprises from about 0.05 to about 0.15% weight percent EDTA. In yet another embodiment, the formulation comprises about 0.1% EDTA.

In another embodiment, the iontophoretic formulation of the present invention comprises about 2 to about 6 weight percent ACV, glycerin and urea wherein the formulation has a pH above 10. In one embodiment, the formulation comprises from about 0.1 to about 0.6 weight percent urea. In another embodiment, the formulation comprises about 0.2% urea.

In an additional embodiment, the iontophoretic formulation of the present invention comprises from about 2 to about 6 weight percent ACV, glycerin and methionine wherein the formulation has a pH above 10. In one embodiment, the methionine is L-methionine. In one embodiment, the formulation comprises from about 0.1 to about 0.6 weight percent methionine. In another embodiment, the formulation comprises about 0.2% weight percent methionine.

In another embodiment, the iontophoretic formulation of the present invention comprises from about 2 to about 6 weight percent ACV, glycerin and benzalkonium chloride wherein the formulation has a pH above 10. In one embodiment, the formulation comprises from about 0.01 weight percent to about 0.03 weight percent benzalkonium chloride.

In a further embodiment, the iontophoretic formulation comprises from about 2 to about 6 weight percent ACV, glycerin, sodium sulfite, EDTA, urea and methionine wherein the formulation has a pH above 10. In another embodiment, the formulation is a gel. In yet another embodiment, the iontophoretic formulation comprises from about 2 to about 6 weight percent ACV, from about 0.05 to 0.15% weight percent EDTA, from about 0.1 to about 0.6 weight percent urea, from about 0.1 to about 0.6 weight percent methionine and from about 0.01 weight percent to about 0.03 weight percent benzalkonium chloride, wherein the formulation has a pH above 10.

In a further embodiment, the iontophoretic formulation comprises about 5 weight percent ACV, glycerin, about 0.1 weight percent EDTA, about 0.2 weight percent urea, about 0.2 weight percent L-methionine and about 0.02 weight percent benzalkonium chloride wherein the formulation has a pH above 10.

The solubility of neutral, unionized acyclovir is very poor. At room temperature and neutral pH the solubility of acyclovir (pKa 2.27 and 9.25) in water is 1.3 mg/mL. Even in an optimized propylene glycol water solution the solubility is only 3 mg/mL. The solubility of unionized acyclovir in the cream formulation, at neutral pH, is also 3 mg/mL. Although the 5% acyclovir cream is formulated to contain 5 weight percent of acyclovir, the bulk of the acyclovir is in the form of a crystalline solid which does not contribute to delivery. An aqueous propylene glycol solution containing 0.3 wt % acyclovir (the continuous phase of the cream) provides nearly the same delivery as the 5% cream itself.

Sodium acyclovir has excellent solubility in water, >100 mg/mL. However, solutions of sodium acyclovir in water alone freeze and precipitate sodium acyclovir on cooling. A variety of water/cosolvent solutions of sodium acyclovir were prepared by neutralizing acyclovir with 1 equivalent of sodium hydroxide. The solubility of sodium acyclovir exceeds 5.7% in any mixture of glycerin and water or propylene glycol and water from 30 to 70% both at room temperature and at 5° C. The solubility of acyclovir (neutral molecule) is <0.5% in all of the cases above.

In a preferred embodiment, it is important to almost completely neutralize the acyclovir in order to avoid precipitation of the neutral molecule in the preparation of sodium acyclovir solutions. For example in the preparation of a 5% solution of sodium acyclovir if the acyclovir is only 90% neutralized, 0.5% acyclovir (neutral) may be present in the solution. But this solution will be relatively unstable as a small temperature change or small amount of evaporation will result in the precipitation of acyclovir (neutral).

A preferred approach is to add approximately one equivalent or a slight excess of base and confirm that the pH is in the expected range. Monitoring pH during the neutralization of acyclovir with a base like sodium hydroxide may not be adequate because small pH changes are associated with large changes in the concentration of sodium acyclovir.

Other preferred embodiments are set forth in the Table 4 below:

TABLE 4

| | sample no.: | | | | | | | |
|---|---|---|---|---|---|---|---|---|
| | NB2000-26A | NB2000-26A | NB2000-28B | NB2000-28B | NB2000-29C | NB2000-29C | NB2000-30D | NB2000-30D |
| | grams | % | grams | % | grams | % | grams | % |
| H2O | 276.13 | 91.42 | 271.48 | 90.17 | 124.69 | 41.46 | 118.91 | 39.59 |
| Glycerin | 0 | 0 | 0 | 0 | 150 | 49.88 | 150 | 49.94 |
| Na2HPO4 | 0 | 0 | 2.407 | 0.80 | 0 | 0 | 2.407 | 0.80 |
| Na3PO4 | 0 | 0 | 4.051 | 1.35 | 0 | 0 | 4.055 | 1.35 |
| Acyclovir | 12.04 | 3.99 | 12.03 | 4.00 | 12.01 | 3.99 | 12.01 | 4.00 |
| 5.0 N NaOH | 11.79 | 3.90 | 9.037 | 3.00 | 12.613 | 4.19 | 11.334 | 3.77 |
| HEC 250 | 2.10 | 0.70 | 2.08 | 0.69 | 1.205 | 0.40 | 1.201 | 0.40 |
| HHX | | | | | | | | |
| 18.5% HCl | 0 | 0 | 0 | 0 | 0.211 | 0.07 | 0.468 | 0.16 |
| pH | | 11.02 | | 11.02 | | 11.00 | | 10.96 |
| viscosity | | 625 | | 672 | | 555 | | 590 |
| (cP)* | | | | | | | | |
| total | 302.06 | 100.00 | 301.09 | 100.00 | 300.73 | 100.00 | 300.39 | 100.00 |

*Brookfield spindle #3 at 20 rpm at 25 C.

The formulations of the invention are further characterized by good to excellent stability. That is, the appearance of the formulation (color, transparency, etc.) remains substantially constant over the period of three to seven days at 5° C. as shown in Table 5.

TABLE 5
Acyclovir Gels (A–L) - May 11, 2005
Storage Temperature = 5 C.

| Sample | Solvent system | ACV conc. (%) | base | pH | HEC (%) | buffer | Stable (Y/N) |
|---|---|---|---|---|---|---|---|
| A | H20:PVP-k17 (98:2) | 4:7 | NaOH | 11.0 | 1.0 | none | No |

t = 0: clear, colorless.
t = 1 day: transparent with numerous white, fibrous-like patterns throughout sample.
t = 3 days: transparent with white, fibrous material at botom of sample.
t = 7 days: transparent with white, fibrous material at botom of sample.

| | | | | | | | |
|---|---|---|---|---|---|---|---|
| B | Sorbitol:H20 (67:33) | 4.7 | NaOH | 11.0 | 1.0 | none | Yes |

t = 0: transparent, slight haze, very light tan color.
t = 1 day: transparent, slight haze, very light tan color.
t = 3 days: transparent, slight haze, very light tan color.
t = 7 days: transparent, slight haze, very light tan color.

| | | | | | | | |
|---|---|---|---|---|---|---|---|
| C | Sorbitol:H20 (67:33) | 4.7 | NaOH | 11.0 | 1.0 | Phosphate | Yes |

t = 0: transparent, slight haze, light tan color.
t = 1 day: transparent, slight haze, light tan color.
t = 3 days: transparent, slight haze, light tan color.
t = 7 days: transparent, slight haze, light tan color.

| | | | | | | | |
|---|---|---|---|---|---|---|---|
| D | Glycerin:H20 (1:1) | 4.7 | NaOH | 11.0 | 1.0 | none | Yes |

t = 0: transparent, slight haze, colorless.
t = 1 day: transparent, slight haze, colorless.
t = 3 days: transparent, slight haze, colorless.
t = 7 days: transparent, slight haze, colorless.

| | | | | | | | |
|---|---|---|---|---|---|---|---|
| E | Sorbitol:H20 (67:33) | 2.1 | NaOH | 10.5 | 1.0 | none | Yes |

t = 0: transparent, slight haze, very light tan color.
t = 1 day: transparent, slight haze, very light tan color.
t = 3 days: transparent, slight haze, very light tan color.
t = 7 days: transparent, slight haze, very light tan color.

| | | | | | | | |
|---|---|---|---|---|---|---|---|
| F | Glycerin:H20 (1:1) | 1.5 | NaOH | 10.5 | 1.0 | none | Yes |

t = 0: transparent, slight haze, colorless.
t = 1 day: transparent, slight haze, colorless.
t = 3 days: transparent, slight haze, colorless.
t = 7 days: transparent, slight haze, colorless.

| | | | | | | | |
|---|---|---|---|---|---|---|---|
| G | Glycerin:H20 (1:1) | 1.5 | NaOH | 10.5 | 1.0 | Phosphate | No |

t = 0: transparent, slight haze, colorless.
t = 1 day: transparent, slight haze, colorless.
t = 3 days: transparent, white particles suspended throughout sample; colorless.
t = 7 days: transparent, large white particles suspended throughout sample; colorless.

| | | | | | | | |
|---|---|---|---|---|---|---|---|
| H | Glycerin:H20 (1:1) | 0.8 | NaOH | 10 | 1 | none | No |

t = 0: transparent, slight haze, colorless.
t = 1 day: transparent with numerous white particles suspended throughout sample; colorless.
t = 3 days: translucent with numerous white particles suspended throughout sample; colorless.
t = 7 days: translucent with numerous white particles suspended throughout sample; colorless.

| | | | | | | | |
|---|---|---|---|---|---|---|---|
| I | Glycerin:H20 (1:1) | 0.8 | TEA | 10.0 | 1.0 | none | No |

t = 0: transparent, slight haze, colorless.
t = 1 day: Opaque, white with uniform suspension of white particles throughout sample; colorless.
t = 3 days: Opaque, white with uniform suspension of white particles throughout sample; colorless.
t = 7 days: Opaque, white with uniform suspension of white particles throughout sample; colorless.

| | | | | | | | |
|---|---|---|---|---|---|---|---|
| J | Sorbitol:H20 (67:33) | 0.5 | NaOH | 10.0 | 1.0 | none | Yes |

t = 0: translucent, moderate haze, very very light tan color.
t = 1 day: translucent, moderate haze, very very light tan color.
t = 3 days: translucent, moderate haze, very very light tan color.
t = 7 days: translucent, moderate haze, very very light tan color.

| | | | | | | | |
|---|---|---|---|---|---|---|---|
| K | Glycerin:H20 (1:1) | 0.4 | NaOH | 9.5 | 1.0 | none | No |

t = 0: transparent, slight haze, colorless.
t = 1 day: translucent having uniform suspension of white particles throughout sample; colorless.
t = 3 days: translucent having uniform suspension of white particles throughout sample; colorless.
t = 7 days: translucent having uniform suspension of white particles throughout sample; colorless.

| | | | | | | | |
|---|---|---|---|---|---|---|---|
| L | Glycerin:H20 (1:1) | 0.4 | TEA | 9.5 | 1.0 | none | No |

t = 0: transparent, slight haze, colorless.
t = 1 day: transparent, slight haze, colorless.
t = 3 days: transparent. Uniform dispersion of white particles; colorless.
t = 7 days: transparent. Uniform dispersion of white particles; colorless.

The invention further comprises methods of treating a viral infection in a target tissue of a patient comprising iontophoretically delivering a formulation of the invention to the infected target tissue of the patient. Viral infections include, but are not limited to, herpetic symptoms and recurrent herpetic symptoms, including lesions (oral or genital) and Varicella zoster i.e., shingles. The patient is preferably a human patient in need of antiviral treatment of a target tissue.

Preferred iontophoretic delivery devices useful with the compositions and methods of the invention include but are not limited to those described in U.S. Pat. Nos. 6,148,231, 6,385,487, 6,477,410, 6,553,253, and U.S. Patent Publication Numbers 2004/0111051, 2003/0199808, 2004/0039328, 2002/0161324, and U.S. Application Ser. No. 60/743,528, all incorporated herein by reference. A preferred applicator which has been developed for use with a device for electrokinetically delivering a medicament to a treatment site comprising an applicator head having opposite faces and including an active electrode and a porous pad (such as a woven or non-woven polymer, for example, a polypropylene pad); a margin of the applicator head about the active electrode having a plurality of spaced projections there along; the porous pad and the applicator head being ultrasonically welded to one another about the margin of the head with the electrode underlying the porous pad; and a medicament or a medicament and an electrically conductive carrier therefor carried by the porous pad in electrical contact with the electrode. Alternatively or additionally, the applicator has been developed for use with a device for electrokinetically delivering a medicament to a treatment site comprising an applicator head having opposite faces and including an active electrode and a porous pad overlying the active electrode; a medicament or a medicament and an electrically conductive carrier therefor carried by the pad and in electrical contact with the electrode; a lid overlying the porous pad on a side of the porous pad remote from the electrode and releasably secured to the applicator head; and the lid comprising layers of different materials and including one or more tabs, one of the layers of the lid and the tab being formed of a metallic material, at least a portion of an interface between the metallic material of the tab and the metallic material of the lid having a discontinuity. In another embodiment, the lid may be an oversized disc having a rim constituting an annular tab. Additionally or alternatively, the applicator which has been developed for use with a device for electrokinetically delivering a medicament to a treatment site comprising an applicator head having opposite first and second faces and including an active electrode and a porous pad overlying said electrode; a medicament or a medicament and an electrically conductive carrier therefor carried by the pad; a margin of the cartridge about the active electrode and a margin of the porous pad being secured to one another; the active electrode having a first portion thereof exposed through the first face of the applicator head remote from the porous pad; and another portion of the active electrode being exposed to the porous pad along the second face of the applicator head for electrical contact with the medicament or the medicament and the electrically conductive carrier.

In yet another embodiment, the stable formulations can be administered topically without the aid of an iontophoretic device.

The following Experiments further illustrate the present invention but should not be construed as in any way limiting its scope.

### EXPERIMENTAL

### Example 1

### Characterization of Acyclovir Sodium Gels

Thirty-two 5.7% acyclovir sodium gels were prepared by weight with varying amounts of glycerin or propylene glycol (30, 40, 50, 60 or 70%). Each of these base acyclovir sodium gels was neutralized stoichiometrically at levels of 88, 105, and 116% with respect to acyclovir. These acyclovir sodium gels were thickened using 0.40% Natrosol 250 HHX (hydroxyethyl cellulose) and contained no sodium phosphate. Two additional acyclovir sodium gels were prepared at the 50% solvent and 105% neutralization level, one with glycerin and the other with propylene glycol. To these two acyclovir sodium gels, 0.80% sodium phosphate dibasic and 1.35% sodium phosphate tribasic dodecahydrate were added. These also included 0.40% Natrosol 250 HHX as a thickener. The acyclovir sodium gels prepared were rotated overnight to ensure good mixing prior to any analysis. The appearance of each finished acyclovir sodium gel can be found in Table 6 below.

TABLE 6

| Acyclovir | | |
|---|---|---|
| Sodium Gel | Characteristics | Appearance |
| 1 | 5.7% ACV, 30% Glycerin, 88% neutralization level | opaque, white solution |
| 2 | 5.7% ACV, 30% Glycerin, 105% neutalization level | clear, colorless solution |
| 3 | 5.7% ACV, 30% Glycerin, 116% neutralization level | clear, colorless solution |
| 4 | 5.7% ACV, 40% Glycerin, 88% neutralization level | opaque, white solution |
| 5 | 5.7% ACV, 40% Glycerin, 105% neutralization level | clear, colorless solution |
| 6 | 5.7% ACV, 40% Glycerin, 116% neutralization level | clear, colorless solution |
| 7 | 5.7% ACV, 50% Glycerin, 88% neutralization level | opaque, white solution |
| 8 | 5.7% ACV, 50% Glycerin, 105% neutralization level | clear, colorless solution |
| 9 | 5.7% ACV, 50% Glycerin, 116% neutralization level | clear, colorless solution |
| 10 | 5.7% ACV, 60% Glycerin, 88% neutralization level | opaque, white solution |
| 11 | 5.7% ACV, 60% Glycerin, 105% neutralization level | clear, colorless solution |
| 12 | 5.7% ACV, 60% Glycerin, 116% neutralization level | clear, colorless solution |
| 13 | 5.7% ACV, 70% Glycerin, 88% neutralization level | opaque, white solution |
| 14 | 5.7% ACV, 70% Glycerin, 105% neutralization level | clear, colorless solution |
| 15 | 5.7% ACV, 70% Glycerin, 116% neutralization level | clear, colorless solution |
| 16 | 5.7% ACV, 30% PG, 88% neutralization level | opaque, white solution |
| 17 | 5.7% ACV, 30% PG, 105% neutralization level | clear, colorless solution |
| 18 | 5.7% ACV, 30% PG, 116% neutralization level | clear, colorless solution |
| 19 | 5.7% ACV, 40% PG, 88% neutralization level | opaque, white solution |
| 20 | 5.7% ACV, 40% PG, 105% neutralization level | clear, colorless solution |
| 21 | 5.7% ACV, 40% PG, 116% neutralization level | clear, colorless solution |
| 22 | 5.7% ACv, 50% PG, 88% neutralization level | opaque, white solution |
| 23 | 5.7% ACV, 50% PG, 105% neutralization level | clear, colorless solution |
| 24 | 5.7% ACV, 50% PG, 116% neutralization level | clear, colorless solution |
| 25 | 5.7% ACV, 60% PG, 88% neutralization level | opaque, white solution |
| 26 | 5.7% ACv, 60% PG, 105% neutralization level | clear, colorless solution |
| 27 | 5.7% ACV, 60% PG, 116% neutralization level | clear, colorless solution |
| 28 | 5.7% ACV, 70% PG, 88% neutralization level | opaque, white solution |
| 29 | 5.7% ACV, 70% PG, 105% neutralization level | clear, colorless solution |
| 30 | 5.7% ACV, 70% PG, 116% neutralization level | clear, colorless solution |
| 31 | 5.7% ACV, 50% Glycerin, 105% neutralization level | clear, colorless solution |
| | with 0.80% sodium phosphate dibasic and 1.35% | |
| | sodium phophate tribasic dodecahydrate | |
| 32 | 5.7% ACV, 50% PG, 105% neutralization level with | cloudy, colorless solution |
| | 0.80% sodium phosphate dibasic and 1.35% sodium | |
| | phosphate tribasic dodecahydrate | |

The 32 acyclovir sodium gels were analyzed by HPLC to determine the amount of soluble acyclovir in each gel. A 1 ml aliquot of each acyclovir sodium gel was transferred to a microcentrifuge tube and spun for 5 minutes at 13,200 RPM. The presence or absence of a pellet was noted and an aliquot of the supernatant was removed for dilution and analysis by HPLC. The pH and conductivity of each acyclovir sodium gel was measured. The Table 7 below provides a summary of the results for the analysis of each acyclovir sodium gel.

TABLE 7

| | | | | | | Estimated | Calculated | Measured |
|---|---|---|---|---|---|---|---|---|
| | Theoretical | Calculated | | | | Form | Form | HPLC |
| | % ACV | % ACV | | | Conductivity | Density | Cone | Sample Cone |
| Acyclovir Sodium Gel | (w/w) | (w/w) | Precipitate | pH | (mS/cm) | (g/ml) | (mg/ml) | (mg/ml) |
| 30% Glycerin/88% neutralized | 5.70% | 5.37% | yes | 11.04 | 8.09 | 1.07 | 57.46 | 0.0345 |
| 30% Glycerin/105% neutralized | 5.69% | 5.79% | no | 11.72 | 6.31 | 1.07 | 61.93 | 0.0372 |
| 30% Glycerin/116% neutralized | 5.70% | 5.80% | no | 12.10 | 7.76 | 1.07 | 62.01 | 0.0372 |
| 40% Glycerin/88% neutralized | 5.70% | 5.29% | yes | 11.02 | 3.50 | 1.10 | 58.18 | 0.0349 |
| 40% Glycerin/105% neutralized | 5.66% | 5.73% | no | 11.61 | 4.28 | 1.10 | 63.04 | 0.0378 |
| 40% Glycerin/116% neutralized | 5.71% | 5.76% | no | 11.97 | 5.35 | 1.10 | 63.35 | 0.0380 |
| 50% Glycerin/88% neutralized | 5.70% | 5.25% | yes | 10.96 | 2.202 | 1.13 | 59.38 | 0.0356 |
| 50% Glycerin/105% neutralized | 5.69% | 5.76% | no | 11.46 | 2.714 | 1.13 | 65.11 | 0.0391 |
| 50% Glycerin/116% neutralized | 5.70% | 5.77% | no | 11.80 | 3.41 | 1.13 | 65.18 | 0.0391 |
| 60% Glycerin/88% neutralized | 5.70% | 5.25% | yes | 10.94 | 1.249 | 1.16 | 60.90 | 0.0365 |
| 60% Glycerin/105% neutralized | 5.70% | 5.68% | no | 11.41 | 1.565 | 1.16 | 65.87 | 0.0395 |
| 60% Glycerin/116% neutralized | 5.70% | 5.69% | no | 11.72 | 1.929 | 1.16 | 65.97 | 0.0396 |
| 70% Glycerin/88% neutralized | 5.69% | 5.19% | yes | 10.90 | 0.575 | 1.18 | 61.28 | 0.0368 |
| 70% Glycerin/105% neutralized | 5.71% | 5.78% | no | 11.32 | 0.675 | 1.18 | 68.25 | 0.0409 |
| 70% Glycerin/116% neutralized | 5.69% | 5.71% | no | 11.63 | 0.908 | 1.18 | 67.42 | 0.0405 |
| 30% PG/88% neutralized | 5.70% | 5.48% | yes | 11.07 | 4.23 | 1.02 | 55.93 | 0.0336 |
| 30% PG/105% neutralized | 5.70% | 5.69% | no | 12.24 | 5.45 | 1.02 | 58.04 | 0.0345 |
| 30% PG/116% neutralized | 5.70% | 5.88% | no | 12.61 | 7.19 | 1.02 | 59.46 | 0.0357 |
| 40% PG/88% neutralized | 5.69% | 5.18% | yes | 11.08 | 2.896 | 1.03 | 53.30 | 0.0320 |
| 40% PG/105% neutralized | 5.70% | 5.53% | no | 12.27 | 3.80 | 1.03 | 56.92 | 0.0342 |
| 40% PG/116% neutralized | 5.70% | 5.38% | no | 12.63 | 4.88 | 1.03 | 55.42 | 0.0333 |
| 50% PG/88% neutralized | 5.69% | 5.01% | yes | 11.16 | 1.927 | 1.03 | 51.65 | 0.0310 |
| 50% PG/105% neutralized | 5.71% | 5.81% | no | 12.34 | 2.503 | 1.03 | 50.89 | 0.0359 |
| 50% PG/116% neutralized | 5.72% | 5.61% | no | 12.68 | 3.25 | 1.03 | 57.74 | 0.0346 |
| 60% PG/88% neutralized | 5.71% | 5.29% | yes | 11.20 | 1.252 | 1.04 | 55.05 | 0.0330 |
| 60% PG/105% neutralized | 5.71% | 5.60% | no | 12.33 | 1.566 | 1.04 | 58.25 | 0.0349 |
| 60% PG/116% neutralized | 5.69% | 5.55% | no | 12.67 | 2.023 | 1.04 | 57.76 | 0.0347 |
| 70% PG/88% neutralized | 5.70% | 5.41% | yes | 11.30 | 0.711 | 1.04 | 56.27 | 0.0338 |
| 70% PG/105% neutralized | 5.71% | 5.47% | no | 12.37 | 0.931 | 1.04 | 56.87 | 0.0341 |
| 70% PG/116% neutralized | 5.70% | 5.69% | no | 12.69 | 1.189 | 1.04 | 59.17 | 0.0355 |
| 50% Glycerin/105% neutralized | 5.69% | 5.79% | no | 11.22 | 3.66 | 1.13 | 65.46 | 0.0393 |
| w/Na Phos | | | | | | | | |
| 50% PG/105% neutralized | 5.70% | 5.88% | yes | 11.26 | 2.244 | 1.03 | 60.55 | 0.0363 |
| w/Na Phos | | | | | | | | |

In all the cases of complete acyclovir neutralization (105 and 116 mole % sodium hydroxide), all the acyclovir was in solution. Thus the solubility of acyclovir sodium in these solutions is greater than 5.7 weight percent. In the case of partial neutralization (88 mole % sodium hydroxide) in the glycerin solutions, the soluble acyclovir was 5.3%. This result is the sum of the soluble sodium acyclovir, 5.0% based on the base charge, plus soluble neutral acyclovir, 0.3% calculated by difference. Essentially the same values are obtained from the propylene glycol/water formulations. The solubility of neutral acyclovir in the acyclovir sodium gels is similar to the solubility observed in propylene glycol/water solutions at neutral pH. The observed pH of the 88% neutralized solutions is weakly affected by the cosolvent to water ratio. The observed pH in the propylene glycol system, 11.2, is slightly higher than the observed pH in the glycerin/water system, 11.0. The pKa of acyclovir as an acid is reported to be 9.25 in dilute aqueous solution. Using the ratio of sodium acyclovir to acyclovir (5.0/0.3) and the pH value of the glycerin/water solution (11.0), the apparent pKa of acyclovir in the glycerin formulation is 9.8.

Experimental Protocol:

### Example 2

### In Vitro Iontophoretic Delivery of Acyclovir through Nude Rat Skin

The formulation was thoroughly mixed and a sufficient amount (about 2 ml) of formulation is syringed out and slowly injected into the drug cartridge pad. The drug cartridge has previously been described in U.S. Pat. Nos. 6,148,231, 6,385,487, 6,477,410, 6,553,253, and U.S. Patent Publication Numbers 2004/0111051, 2003/0199808, 2004/0039328, 2002/0161324, all incorporated herein by reference. After the drug cartridge pads were prepared they were pressed with gloved finger to distribute the formulation evenly in the pad. Target weight in the drug cartridge was 160-200 mg.

Freshly excised skin from hairless rat was mounted on Franz diffusion cells, such that the stratum corneum side of the skin faced the donor compartment of the cell. Cells were connected in series to a constant current power supply and a current of 0.2 mA/cm² (0.13 mA over surface area of 0.64 cm²) was applied. The samples were analyzed by HPLC. In vivo iontophoretic delivery in rabbits with analysis by microdialysis showed unexpectedly that acyclovir in glycerin (5% 29 C) penetrates the skin approximately five fold better than acyclovir in propylene glycol (5% 29 C-PG).

The methods described for the microdialysis study are described in Stagni et al., supra, which is incorporated herein by reference.

### Example 3

### Increasing the Stability of the ACV in Formulation

An analysis of the degradation of 5% ACV in glycerin formulations was conducted by HPLC. Six degradants were identified. Peaks corresponding to these degradants were believed to represent additions to the acyclovir molecule by oxidative degradants of glycerin.

It was next determined whether it was possible to increase the stability of 5% ACV formulations by adding varying amounts of the additives designated in the following Table were tested by storing each sample for 4 weeks at 40° C. The percent intact ACV remaining in the formulations after 4 weeks is shown in the Table below.

TABLE

| Sodium | | | | |
|---|---|---|---|---|
| sulfite (%) | EDTA (%) | Urea (%) | Methionine (%) | ACV area (%) |
| 0 | 0.1 | 0.25 | 0.5 | 99.9 |
| 0 | 0.05 | 0.25 | 0.25 | 99.89 |
| 0 | 0 | 0.5 | 0.5 | 99.89 |
| 0 | 0 | 0.5 | 0.5 | 99.89 |
| 0 | 0.05 | 0 | 0.5 | 99.88 |
| 0 | 0.1 | 0 | 0.25 | 99.87 |
| 0.25 | 0.1 | 0.5 | 0.5 | 99.86 |
| 0.25 | 0.05 | 0.25 | 0.5 | 99.86 |
| 0 | 0 | 0 | 0 | 95.85 |

### As shown in the above Table, the control formulation containing only 5% ACV had decreased stability compared to formulations comprising the additives, sodium sulfite, EDTA, urea and/or methionine.

The stability of a formulation comprising 5% ACV as a gel at pH 11 containing 0.1% EDTA, 0.2% urea, 0.2% L-methionine and 0.02% benzalkonium chloride was additionally tested by storing the formulation for 8 weeks at 40° C. After 8 weeks, the formulation comprised 99.8% non-degraded ACV. As such, it was determined that this formulation resulted in minimal degradation.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

As used in this specification and the appended claims, the singular forms “a”, “an”, and “the” include the plural, unless the context clearly dictates otherwise.

While this invention has been particularly shown and described with references to preferred embodiments thereof, it will be understood by those skilled in the art that various changes in form and details may be made therein without departing from the scope of the invention encompassed by the appended claims.

What is claimed:

1. A viscous formulation suitable for iontophoresis comprising acyclovir and a pharmaceutically acceptable carrier or excipient, wherein the pH of the formulation is at least about 10.

2. The formulation of claim 1 wherein the viscosity of the formulation is no less than about 500 cp at 25° C.

3. The formulation of claim 1 wherein the formulation comprises a viscosity modulating agent selected from, cellulosic polymers and derivatives thereof, crosslinked acrylic acid polymers, hydrophilic polymers, gums, sodium alginate, gelatin and any combination thereof.

4. The formulation of claim 1 comprising 0.1 to 10 weight percent of a viscosity modulating agent.

5. The formulation of claim 1 comprising a hydrating agent.

6. The formulation of claim 5 wherein the hydrating agent is glycerin.

7. The formulation of claim 6 wherein the composition is substantially free of buffer.

8. The formulation of claim 1 comprising water.

9. The formulation of claim 8 comprising from about 20 to about 80 weight percent of water.

10. The formulation of claim 1 comprising a buffer.

11. The formulation of claim 1 further comprising one or more additives selected from the group consisting of an antioxidant, a stabilizer, a chelator, a preservative and an aldehyde scavenger.

12. The formulation of claim 10 comprising from about 10 to about 80 weight percent of glycerin.

13. The formulation of claim 1 comprising a base.

14. The formulation of claim 13 wherein at least 1 equivalent of base is used in relation to acyclovir.

15. The formulation of claim 14 wherein the base is sodium hydroxide.

16. The formulation of claim 1 comprising acyclovir, glycerin, sodium hydroxide and an etherified cellulose polymer.

17. The formulation of claim 16 further comprising water.

18. The formulation of claim 17 wherein at least 1 equivalent of sodium hydroxide is used in relation to acyclovir.

19. The formulation of claim 1 comprising acyclovir, sorbitol, sodium hydroxide and an etherified cellulose polymer.

20. The formulation of claim 1 consisting essentially of acyclovir, glycerin, sodium hydroxide, water and an etherified cellulose polymer.

21. The formulation of claim 20 wherein at least 1 equivalent of sodium hydroxide is used in relation to acyclovir.

22. The formulation of claim 1 comprising acyclovir, propylene glycol, sodium hydroxide and an etherified cellulose polymer.

23. The formulation of claim 22 further comprising water.

24. The formulation of claim 23 wherein at least 1 equivalent of sodium hydroxide is used in relation to acyclovir.

25. The formulation of claim 1 comprising acyclovir, glycerin, propylene glycol, sodium hydroxide and an etherified cellulose polymer.

26. The formulation of claim 25 further comprising water.

27. The formulation of claim 26 wherein at least 1 equivalent of sodium hydroxide is used in relation to acyclovir.

28. An iontophoretic formulation of claim 1 comprising, about 4 weight percent of acyclovir; about 87 weight percent of sorbitol (70%); about 8 weight percent of sodium hydroxide; and about 1 weight percent of at least one cellulosic polymer.

29. An iontophoretic formulation of claim 1 comprising, about 4 weight percent of acyclovir; about 87 weight percent of sorbitol (70%); about 6 weight percent of 5.0 N sodium hydroxide; about 1 weight percent of at least one cellulosic polymer; about 1 weight percent of Na₂HPO₄; and about 1 weight percent of Na₃PO₄.

30. An iontophoretic formulation of claim 1 comprising about 5 weight percent of acyclovir; about 48 weight percent of glycerin; about 5 weight percent of 5.0 N sodium hydroxide; about 1 weight percent of at least one cellulosic polymer; and about 41 weight percent of water.

31. An iontophoretic formulation of claim 1 comprising, about 2 weight percent of acyclovir; about 93 weight percent of glycerin; about 2 weight percent of 5.0 N sodium hydroxide; about 1 weight percent of at least one cellulosic polymer.

32. An iontophoretic formulation of claim 1 comprising: about 2 weight percent of acyclovir; about 49 weight percent of sorbitol (70%); about 2 weight percent of 5.0 N sodium hydroxide; about 1 weight percent of at least one cellulosic polymer.

33. A formulation suitable for iontophoresis in the form of a solution comprising acyclovir and buffer wherein the pH of the formulation is at least about 10.

34. The formulation of claim 33 wherein the buffer is phosphate buffer.

35. An iontophoretic formulation of claim 1 comprising, about 4 weight percent of acyclovir; about 50 weight percent of glycerin; about 2 weight percent phosphate buffer; about 4 weight percent of 5.0 N sodium hydroxide; about 0.4 weight percent of at least one cellulosic polymer, about 40 weight percent of water.

36. An iontophoretic formulation of claim 1 comprising, about 5 weight percent of acyclovir; about 50 weight percent of glycerin; about 0.9 weight percent of sodium hydroxide; about 0.35 weight percent of at least one cellulosic polymer, about 43.75 weight percent of water.

37. An iontophoretic formulation of claim 1 comprising, about 4 weight percent of acyclovir; about 50 weight percent of glycerin; about 1.78 weight percent of sodium hydroxide; about 0.40 weight percent of at least one cellulosic polymer; about 41.67 weight percent of water and about 2.15 weight percent of sodium phosphate solution.

38. An iontophoretic formulation of claim 1 comprising about 4 weight percent of acyclovir; about 50 weight percent of propylene glycol; about 1.78 weight percent of sodium hydroxide; about 0.40 weight percent of at least one cellulosic polymer, about 43.82 weight percent of water.

39. An iontophoretic formulation of claim 1 comprising from about 2 to about 6 weight percent acyclovir, from about 0.05 to 0.15% weight percent EDTA, from about 0.1 to about 0.6 weight percent urea, from about 0.1 to about 0.6 weight percent L-methionine and from about 0.01 weight percent to about 0.03 weight percent benzalkonium chloride.

40. An iontophoretic formulation of claim 1 comprising about 5 weight percent acyclovir, about 0.1 weight percent EDTA, about 0.2 weight percent urea, about 0.2 weight percent L-methionine and about 0.02 weight percent benzalkonium chloride wherein the formulation has a pH above 10.

41. A formulation according to claim 1 further comprising a porous pad.

42. An iontophoretic device comprising a formulation according to claim 1 absorbed onto a porous pad.

43. A method for treating herpes comprising iontophoretically administering to the body surface of a patient in need thereof, the formulation of claim 1.
